(19) United States
(12) Patent Application Publication
(10) Pub. No.: US 2025/0387331 A1
(43) Pub. Date: Dec. 25, 2025

(54) PROSTAGLANDIN ANALOGS AND METHODS FOR SUSTAINED GLAUCOMA MANAGEMENT

(71) Applicant: Peregrine Ophthalmic PTE LTD, Singapore (SG)

(72) Inventors: Shih-Horng Su, Irvine, CA (US); Tina Howden, Singapore (SG)

(21) Appl. No.: 19/048,696

(22) Filed: Feb. 7, 2025

Related U.S. Application Data
(60) Provisional application No. 63/556,793, filed on Feb. 22, 2024.
(60) Provisional application No. 63/663,051, filed on Jun. 21, 2024.

(51) Int. Cl. A61K 9/1271; A61K 9/00; A61K 31/5575; A61K 47/20; A61P 27/02

(52) U.S. Cl. CPC A61K 9/1271; A61K 9/0019; A61K 9/0048; A61K 31/5575; A61K 47/20; A61P 27/02

(57) ABSTRACT

A method for sustained reduction of intraocular pressure (IOP). The method is accomplished by suprachoroidal injection of a pharmaceutical composition that contains a prostaglandin F2a. The IOP is reduced by at least 20% for a period of 30-60 days following the suprachoroidal injection.

### CROSS REFERENCE TO RELATED APPLICATIONS

This application claims the benefit of and priority to U.S. Provisional Patent Application Ser. No. 63/556,793, filed on Feb. 22, 2024, and U.S. Provisional Patent Application Ser. No. 63/663,051, filed on Jun. 21, 2024. The content of the prior applications is incorporated herein by reference in its entirety.

### BACKGROUND

Intraocular pressure (“TOP”) in the eye is maintained by a continuous flow of aqueous humor produced by the ciliary body. Excess fluid flows out of the eye through the trabecular meshwork. If the outflow is blocked, aqueous humor builds up inside the eye leading to increased IOP and ocular hypertension. The ocular hypertension can damage the optic nerve, resulting in an optic neuropathy and irreversibly impaired vision. This condition, known as glaucoma, affects more than 60 million people worldwide and is the second leading cause of blindness. Increased IOP is the key modifiable risk factor for glaucoma.

Conventional treatments for ocular hypertensive and glaucoma patients include ocular surgery and topical eye drop instillation. These treatment modalities have drawbacks. For example, not all patients are candidates for ocular surgery. Additionally, although topical eye drops are generally considered to be effective, patients' long-term compliance with instillation schedules is a major issue. Ocular hypertension and glaucoma cannot be well controlled if patients do not adhere to the proper topical eye drop instillation schedule.

Topical eye drops contain drugs for controlling IOP which typically act by reducing fluid production by the eye, increasing fluid outflow, or by both mechanisms. Prostaglandin analogues, e.g., latanoprost, are potent drugs which can reduce IOP by increasing aqueous outflow through the uveoscleral pathway.

Typically, only 5% of free drug applied to the corneal epithelium via eye drops successfully penetrates through the cornea. As a result, the amount of drug reaching the aqueous humor often falls below the therapeutically effective concentration. This necessitates repeated administration. Additionally, a substantial portion of the drug can enter the circulation via the conjunctival sac, causing undesirable systemic side effects.

The need exists for methods to administer prostaglandin F2α derivatives that result in sustained reduction of IOP after a single dose without the above drawbacks.

### SUMMARY

To meet the need set forth above, a method is provided for sustained reduction of IOP by administering a pharmaceutical composition via suprachoroidal injection in which the composition includes a prostaglandin F2α.

A particular method is also provided for treating glaucoma by administering an effective amount of a pharmaceutical composition that contains a prostaglandin F2α into an eye of a glaucoma patient by suprachoroidal injection.

Also disclosed is use of a pharmaceutical composition that contains a prostaglandin F2α in a method for treating glaucoma in which the composition is administered by suprachoroidal injection.

The details of one or more embodiments of the invention are set forth in the drawings and description below. Other features, objects, and advantages of the invention will be apparent from the description, from the drawing, and from the claims. All references cited herein are hereby incorporated by reference in their entirety.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The invention description below refers to the accompanying drawings.

FIG. 1A is an Anterior Segment-Optical Coherence Tomography (“AS-OCT”) image of a rabbit eye showing the anatomical location of the ciliary body, supraciliary space, sclera, and choroid.

FIG. 1B is an AS-OCT image of a rabbit eye obtained after suprachoroidal injection at least 4-6 mm posterior from the limbus.

FIG. 2 is a plot of IOP percent reduction from baseline versus time after a single administration on day 0 (DO) of liposomal latanoprost (LipoLAT) by subconjunctival injection or by suprachoroidal injection. OD=oculus dexter, OS=oculus sinister, LipoLAT=latanoprost encapsulated in POPC liposomes.

FIG. 3A is an AS-OCT image of a rabbit eye pre-injection.

FIG. 3B is an AS-OCT image of a rabbit eye post-injection. The arrow indicates the injection site of the liposomal latanoprost formulation into the suprachoroidal space

FIG. 3C. shows an AS-OCT image of a rabbit eye 1 month post-injection of the liposomal latanoprost formulation.

FIG. 4 is a plot of IOP percent reduction from baseline versus time after (i) a single suprachoroidal injection of LipoLAT, (ii) a single control saline injection, and (iii) daily application of latanoprost eye drops. The arrow indicates day 14 when latanoprost eye drops were discontinued.

FIG. 5A shows slit-lamp photographs of a rabbit eye pre-injection.

FIG. 5B shows slit-lamp photographs of a rabbit eye 1 week after suprachoroidal injection of a liposomal latanoprost formulation.

FIG. 5C shows slit-lamp photographs of a rabbit eye 12 weeks after suprachoroidal injection of a liposomal latanoprost formulation.

FIG. 6A shows full field electroretinogram in eyes injected with liposomal latanoprost in the suprachoroidal space at baseline.

FIG. 6B shows full field electroretinogram in eyes injected with liposomal latanoprost in the suprachoroidal space at 12 weeks post-injection.

### DETAILED DESCRIPTION

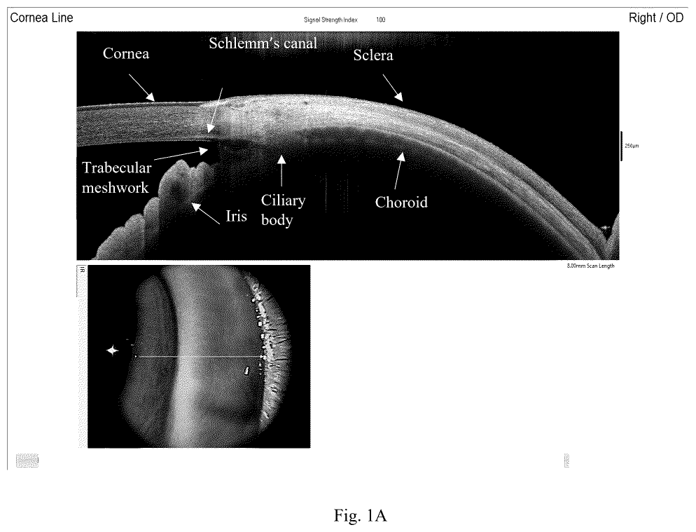

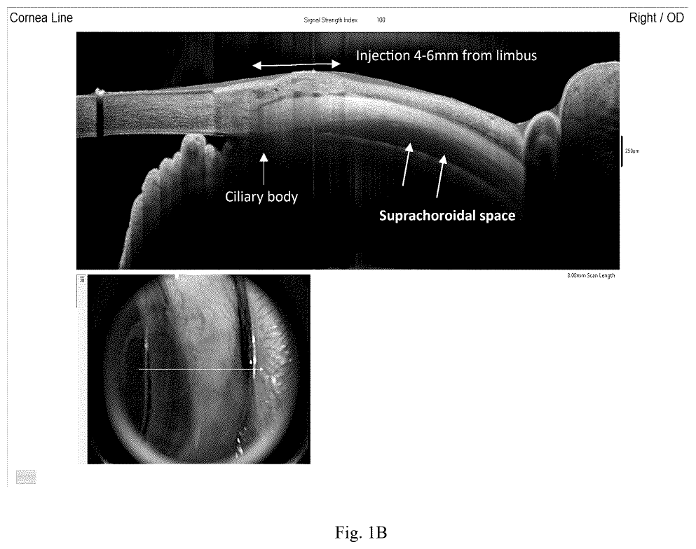

In the method for sustained reduction of IOP set forth above, the pharmaceutical composition is carried out by suprachoroidal injection. The suprachoroidal injection is performed with a needle, preferably a microneedle, through the sclera at least 4-6 mm posterior to the limbus, the border between the cornea and the sclera. FIG. 1A shows the anatomy of a rabbit eye and FIG. 1B shows the injection site. Importantly, the injection location is significantly posterior to the ciliary body. As such, supraciliary injection is excluded from the methods described herein.

The pharmaceutical composition includes a prostaglandin F2α. The prostaglandin F2α can be, but is not limited to, latanoprost, bimatoprost, travoprost, or carboprost. In a specific method, the prostaglandin F2α is latanoprost.

The prostaglandin F2α can be encapsulated in a liposome formed solely of egg phosphatidylcholine (“eggPC”) or palmitoyloleoyl phosphatidyl-choline (“POPC”). In an exemplary method, the prostaglandin F2α is latanoprost and the liposome is formed solely of POPC. Latanoprost-loaded eggPC and POPC liposomes can be those described in U.S. Pat. Nos. 11,452,703; 10,272,040; and 9,956,195. The liposomes can be prepared also as described in these three US Patents.

The concentration of prostaglandin F2α in the pharmaceutical compositions containing liposomes can be 0.5 mg/mL to 3.0 mg/mL (e.g., 0.5, 1.0, 1.5, 2.0, 2.5, and 3.0 mg/mL).

Alternatively, the prostaglandin F2α is not encapsulated in a liposome. Pharmaceutical compositions lacking liposomes contain instead certain pharmaceutically acceptable excipients including, but not limited to, dimethylsulfoxide (“DMSO”), dextran, carboxymethylcellulose, and methylcellulose.

In one pharmaceutical composition, the pharmaceutically acceptable excipient is DMSO. The amount of DMSO in the pharmaceutical composition can be 10% to 30% v/v (e.g., 10%, 15%, 20%, 25%, and 30%). In a particular pharmaceutical composition, the prostaglandin F2α is latanoprost, which can be included at a concentration of 0.01% to 1% w/v (e.g., 0.01%, 0.05%, 0.1%, 0.02%, 0.25%, 0.5%, 0.75%, and 1%), preferably 0.2% to 0.5% w/v. In a specific pharmaceutical composition, the DMSO is present at 10% v/v and the latanoprost is present at 0.2% w/v.

In the method set forth, supra, a single suprachoroidal injection of any of the pharmaceutical compositions described above results in a reduction of IOP by at least 20% for at least 30 to 60 days post-injection. The IOP reduction can be as high as 50% for a period of two weeks following the suprachoroidal injection.

Also mentioned in the SUMMARY section is a method for treating glaucoma by administering by suprachoroidal injection a pharmaceutical composition that contains a prostaglandin F2α.

Similar to the method described above, in the method for treating glaucoma, the prostaglandin F2α can be, but is not limited to, latanoprost, bimatoprost, travoprost, and carboprost.

The pharmaceutical composition can also be the same as those described above.

In a particular method, the pharmaceutical composition includes latanoprost at a concentration of 0.5 mg/mL to 3.0 mg/mL (e.g., 0.5, 1.0, 1.5, 2.0, 2.5, and 3.0 mg/mL) in which the latanoprost is encapsulated in a POPC liposome.

Finally, encompassed by the invention is use of a pharmaceutical composition that contains a prostaglandin F2α in a method for treating glaucoma in which the pharmaceutical composition is administered by suprachoroidal injection. In this use, the prostaglandin F2α can be, but is not limited to, latanoprost, bimatoprost, travoprost, and carboprost.

Without further elaboration, it is believed that one skilled in the art can, based on the description above, utilize the present invention to its fullest extent. The specific example below is to be construed as merely illustrative, and not limitative of the remainder of the disclosure in any way whatsoever.

### Example 1: In Vivo Activity of Latanoprost Liposomal Formulation after Suprachoroidal Injection

Both eyes of 3 Dutch belted rabbits were injected in the study. Baseline IOP was measured for 2 days before injection using a hand-held tonometer. Topical anesthesia was applied to the eye of the non-sedated animals before IOP measurement. Three measurements were taken and the mean IOP was calculated. IOP was measured at the same time to reduce diurnal variability. IOP was measured at 1 h, 4 h, 8 h, and 24 h post-injection, then daily for 14 days, and then weekly until the IOP returned to within 10% of baseline IOP.

A caliper was set to 4 mm and used to measure from the limbus to the desired position for injection. The injection site was typically 4-6 mm from the limbus. See FIG. 1A. A 30 G needle was advanced at the marked position into the sclera to reach the anatomical position of the suprachoroidal space. 150 μL of latanoprost (2 mg/ml) encapsulated in POPC liposomes was slowly injected. AS-OCT imaging post injection confirmed the successful delivery of the drug into the suprachoroidal space. See FIG. 1B. The AS-OCT image clearly showed that the injection was suprachoroidal and not supraciliary, which is much more anterior in location.

For comparison, other rabbits were administered with 150 μL of latanoprost (1 mg/ml) encapsulated in POPC liposomes by subconjunctival injection essentially as described in U.S. Pat. No. 9,956,195.

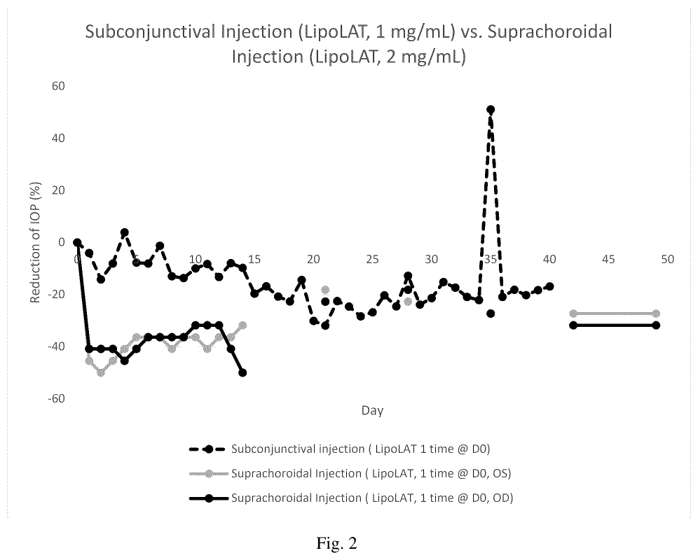

A representative result is shown in FIG. 2. A single suprachoroidal injection of liposomal latanoprost decreased IOP by 40-50% up to 15 days post-injection. By contrast, subconjunctival injection afforded a maximum IOP reduction of 30% at 21 days post-injection. Suprachoroidal injection of latanoprost resulted in a sustained lowering of IOP by at least 20% over the course of the study up to 49 days post-injection.

### Example 2: Safety and Efficacy of Latanoprost Liposomal Formulation for Sustained Release of Latanoprost Injected into the Suprachoroidal Space in the Rabbit Eye

### Injection Technique

A suprachoroidal injector (SupraMedical, Israel) was used that has a 30-gauge needle attached to an extension tubing connected to a 1 ml syringe. The length of the injector is adjusted by inputting readings of scleral thickness combined with a correction factor, supplied on a calibration table from the manufacturer. The scleral thickness was determined by ultrasound bio-microscopy (UBM). After the depth dial was set to the correct position, the plunger of the injector and the syringe were depressed simultaneously. Injections were delivered over 5-10 seconds. After the drug formulation or control was delivered, the plunger was released, and the injector gradually removed.

Rabbits receiving suprachoroidal injection had their scleral thickness measured with Optovue RTVue OCT. The thickness of the sclera was measured at 3 mm posterior to the corneoscleral junction. An average of three readings were taken to determine the mean scleral thickness. The length of the needle on the injector was subsequently calibrated by adding 300 μm to the mean scleral thickness. This additional value was based on the calibration table supplied by the manufacturer. It was required to ensure that the drug was delivered into the suprachoroidal space.

### Surgical Technique

Four Dutch-Belt rabbits (n=8 eyes) were used in this study. Baseline IOP was measured using a calibrated Tonopen XL® (Reichert Ophthalmic Instruments, Depew, NY). Topical anesthesia was not required for IOP measurements. Animals were handled consistently to ensure stable IOP readings. Each rabbit was acclimatized to the IOP measurement procedure for at least 7 days to obtain a stable baseline IOP. Rabbits were divided into three groups. Group A (3 rabbits, n=6 eyes) received a single suprachoroidal injection of 150 μL liposomal latanoprost formulation (latanoprost concentration 2 mg/mL), Group B (1 rabbit, n=2 eyes) received one drop of topical latanoprost (0.005% ophthalmic solution) daily for two weeks, and control Group C (1 rabbit, n=2 eyes) received a single suprachoroidal saline injection. All groups received their treatment at similar time of day, i.e., at 9:00 AM.

All procedures were performed under the microscope under topical anesthesia (0.5% proparacaine HCl) by a single surgeon. The study eye was cleaned with 50% povidone iodine followed by a saline wash. The injector was placed at the 3 o'clock or 9 o'clock position at the limbus, avoiding extraocular muscles. Injection was performed 4-6 mm posterior to limbus. The base of the injector was anchored on the sclera, the needle extended, and 150 μL of liposomal latanoprost or normal saline was injected into the suprachoroidal space. The needle was withdrawn a few seconds later to minimize reflux of the injected drug.

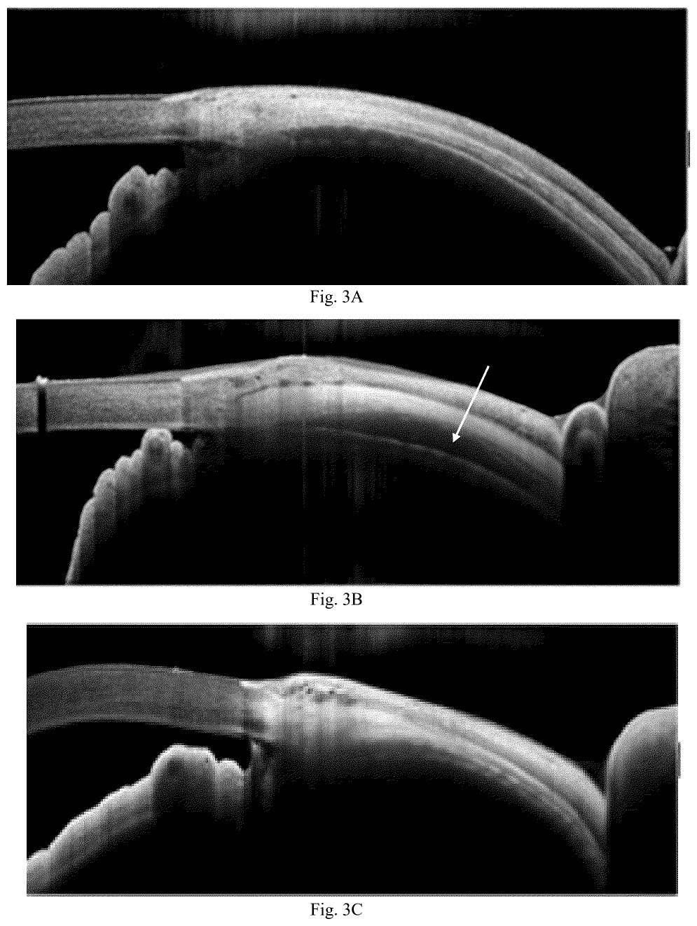

AS-OCT was performed just before and immediately after the procedure to ensure the drug was delivered into the correct space. See FIGS. 3A and 3B. This was confirmed by observing the distension of the suprachoroidal space post-injection. See FIG. 3B. AS-OCT was also performed weeks 1, 4, 8, and 12 weeks post-injection. The AS-OCT showed that the suprachoroidal space closed, indicating that the injected liposomal latanoprost formulation had been diffusely distributed in the eye. As an example, AS-OCT at 4 weeks post-injection demonstrated obliteration of the suprachoroidal space and reattachment of the ciliary body. See FIG. 3C.

The injected eye was given a stat dose of topical tobramycin 3 mg/ml (Tobrex®, Novartis). Procedures were done according to protocols approved by SingHealth Institutional Animal Care and Use Committee (IACUC).

In Group B receiving topical therapy, latanoprost 0.005% ophthalmic solution was delivered into the lower conjunctival sac daily at 9 am for two weeks.

### Statistical Analysis

Statistical analysis included descriptive statistics. Mean and standard deviation (SD) was used for continuous variables whilst percentages were used for categorical variables. A P value of <0.05 was considered statistically significant.

### Results

### Intraocular Pressure

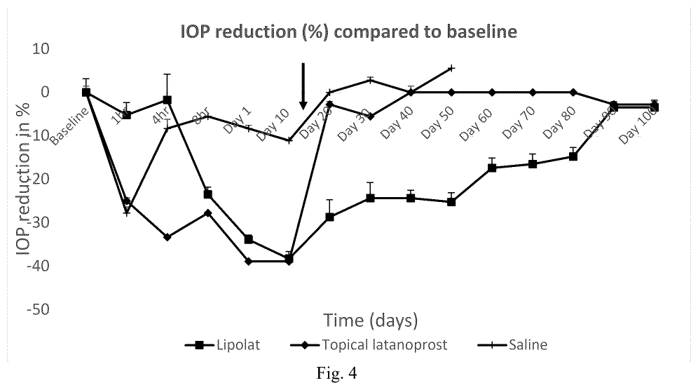

IOP was measured with the Tonopen XL® 1 hour, 4 hours, 8 hours, and 24 hours post procedure. Three readings were taken for each eye and an average obtained. Measurements were then captured every 10 days for month 1, month 2 and month 3. The results are shown in FIG. 4. There was no difference between each of the three treatment groups (liposomal latanoprost, topical latanoprost, saline) in the baseline IOP, measuring 18.7±2.4 mm Hg in all eyes (n=10).

There was a 23% (4.5±1.6 mm Hg) decrease in IOP from baseline at 8 hours post liposomal latanoprost injection, reaching a maximum decrease of 40% (7.7±2.3 mm Hg) decrease from Day 5 to Day 10. The lowered IOP was sustained until about Day 56 before gradually returning to baseline IOP at Day 90.

In comparison, eyes that were administered daily topical latanoprost eye drops showed an acute decrease in IOP of 27% (5.0±0.7 mm Hg) 1 hour post administration, reaching a maximum decrease of 38% and this effect was maintained until the eyedrops were stopped at Day 14. The IOP returned nearly to baseline 6 to 7 days after cessation of the eyedrops.

The control saline-injected group also demonstrated a reduction in IOP of 28% (5±1.4 mm Hg) 1 hour post injection. However, after 4 hours, the IOP promptly started to rise back to baseline. No further IOP decrease was observed thereafter.

### Safety Data

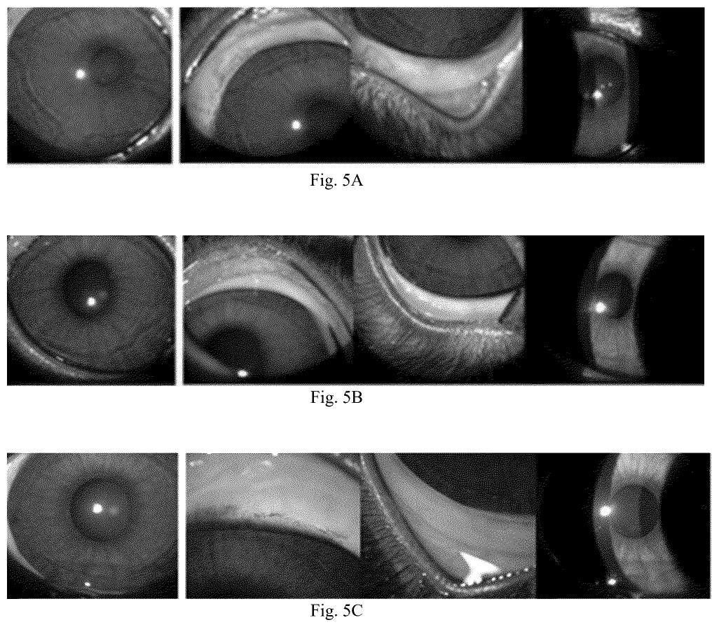

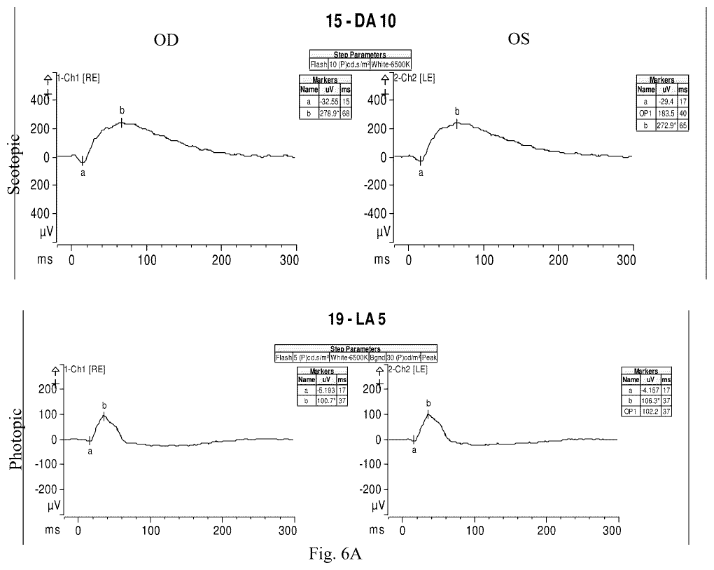

Slit lamp images were obtained pre- and post-injection in all 3 groups. See FIGS. 5A-5C. The conjunctiva were white and quiet. The corneas were clear. There was no evidence of hyperemia, inflammation, or infection at the site of the injection or the surrounding area. This was observed throughout in all three groups up to 12 weeks after the start of the study.

OCT macula were captured at baseline, at week 1, and at 4 week intervals thereafter. The data is shown in Table 1 below.

TABLE 1
OCT retinal thickness over 90 days. Mean (±S.E.M.)
OCT of retinal thickness (μm)of all groups.

| Mean OCT | | | | | | | |
|---|---|---|---|---|---|---|---|
| retinal | | | | | | | |
| thickness | | | | | | Mean | |
| measurement | | | | | Week | retinal | P |
| (μm) | Baseline | Week 1 | Week 4 | Week 8 | 12 | thickness | value |
| LipoLAT | 134.9 ± | 137.9 ± | 135.3 ± | 131.1 ± | 132.8 ± | 134.4 | 0.115 |
| | 5.017 | 4.522 | 6.047 | 4.757 | 6.337 | | |
| Topical | 133.8 ± | 132.7 ± | 131.8 ± | 126.8 ± | 132.1 ± | 131.44 | |
| latanoprost | 3.934 | 3.085 | 4.515 | 6.672 | 4.660 | | |
| Normal | 133.3 ± | 135.3 ± | 133.5 ± | 129.3 ± | | 132.85 | 0.342 |
| Saline | 2.251 | 2.338 | 4.970 | 2.733 | | | |

Retinal thickness remained constant from baseline to week 12 in all treatment groups. Mean retinal thickness was 134.4 μm in the liposomal latanoprost group. Liposomal latanoprost versus topical latanoprost and liposomal latanoprost versus normal saline both showed no significant difference (p=0.115 and p=0.342 respectively) in retinal thickness. P values were calculated using a 2 tailed t-test.

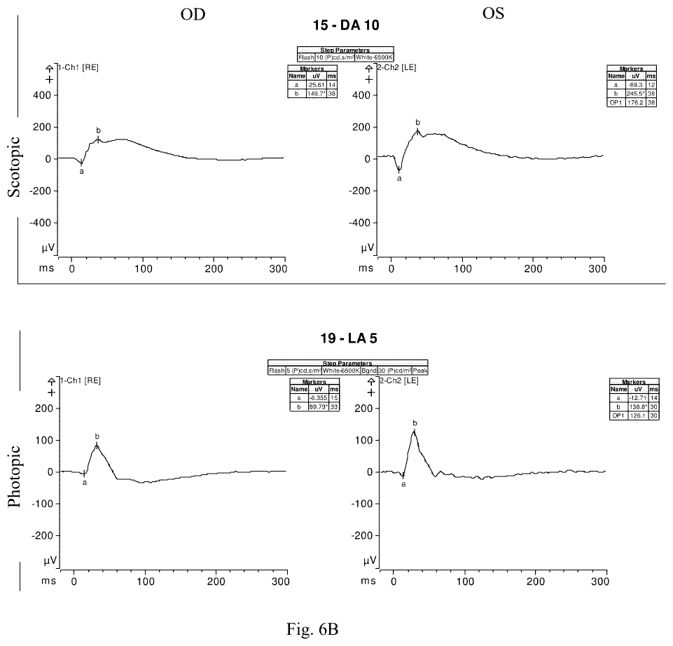

Full field electroretinograms (ERG) were obtained at baseline and at 12 weeks post-injection of liposomal latanoprost. The results are shown in FIG. 6A (baseline) and 6B (12 weeks). No significant differences in retinal function were observed.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A method for sustained reduction of intraocular pressure (IOP), the method comprising administering into an eye of a subject in need thereof an effective amount of a pharmaceutical composition that contains a prostaglandin F2α, wherein the administering is carried out by suprachoroidal injection, and the method excludes supraciliary injection.

2. The method of claim 1, wherein the prostaglandin F2α is selected from the group consisting of latanoprost, bimatoprost, travoprost, and carboprost.

3. The method of claim 2, wherein the prostaglandin F2α is encapsulated in a liposome formed solely of egg phosphatidylcholine or palmitoyloleoyl phosphatidyl-choline (POPC).

4. The method of claim 3, wherein the prostaglandin F2α is latanoprost and the liposome is formed solely of POPC.

5. The method of claim 4, wherein a concentration of latanoprost in the pharmaceutical composition is 0.5 mg/mL to 3.0 mg/mL.

6. The method of claim 1, wherein the pharmaceutical composition further contains a pharmaceutically acceptable excipient.

7. The method of claim 6, wherein the pharmaceutically acceptable excipient is dimethyl sulfoxide (DMSO).

8. The method of claim 7, wherein the pharmaceutical composition includes 10% to 30% v/v DMSO.

9. The method of claim 8, wherein the prostaglandin F2 is latanoprost and a concentration of latanoprost in the pharmaceutical composition is 0.01% to 1% w/v.

10. The method of claim 6, wherein the pharmaceutically acceptable excipient is dextran, carboxymethylcellulose, or methylcellulose.

11. The method of claim 1, wherein the IOP is reduced by at least 20% for a period of 30-60 days following the suprachoroidal injection.

12. A method for treating glaucoma, the method comprising administering into an eye of a subject in need thereof an effective amount of a pharmaceutical composition that contains a prostaglandin F2α, wherein the administering is carried out by suprachoroidal injection, and the method excludes supraciliary injection.

13. The method of claim 12, wherein the prostaglandin F2 is selected from the group consisting of latanoprost, bimatoprost, travoprost, and carboprost.

14. The method of claim 12, wherein the prostaglandin F2α is encapsulated in a liposome formed solely of egg phosphatidylcholine or palmitoyloleoyl phosphatidyl-choline (POPC).

15. The method of claim 14, wherein the prostaglandin F2α is latanoprost and the liposome is formed solely of POPC.

16. The method of claim 15, wherein a concentration of latanoprost in the pharmaceutical composition is 0.5 mg/mL to 3.0 mg/mL.

17. The method of claim 12, wherein the pharmaceutical composition further contains a pharmaceutically acceptable excipient.

18. The method of claim 17, wherein the pharmaceutically acceptable excipient is dimethyl sulfoxide (DMSO).

19. The method of claim 18, wherein the pharmaceutical composition includes 10% to 30% v/v DMSO.

20. The method of claim 19, wherein the prostaglandin F2α is latanoprost and a concentration of latanoprost in the pharmaceutical composition is 0.01% to 1% w/v.
